(19) United States
(12) Patent Application Publication
(10) Pub. No.: US 2014/0314845 A1
(43) Pub. Date: Oct. 23, 2014

(54) SPHERICAL ERYTHRITOL GRANULES FOR DIRECT COMPRESSION AND PROCESS OF PRODUCING SAME, PROCESS OF PRODUCING TABLETS BY TABLETING THE ERYTHRITOL GRANULES WITH A PHARMACEUTICALLY EFFECTIVE INGREDIENT OR NUTRITIONAL INGREDIENT, AND TABLETS OR ORDINARY CANDY TABLETS OBTAINED THEREBY

(71) Applicant: Toshikatsu YAMANAKA, Gunma (JP)

(72) Inventor: Toshikatsu YAMANAKA, Gunma (JP)

(73) Assignee: MICROFOODS JAPAN, CO., LTD., Gunma (JP)

(21) Appl. No.: 13/928,122

(22) Filed: Jun. 26, 2013

(30) Foreign Application Priority Data
Apr. 19, 2013 (JP) JP2013-088712

(51) Int. Cl. A61K 9/16; A61K 45/06; B02C 23/14; A61K 31/047

(52) U.S. Cl. CPC A61K 9/1682; A61K 31/047; A61K 45/06; B02C 23/14
USPC 424/464; 568/852; 514/738; 241/24.1

(57) ABSTRACT

It was impossible to prepare a granular product composed only of fine erythritol powders by using conventional erythritol having a particle diameter on the order of 100 μm. Spherical erythritol granules for direct compression obtained by granulating, drying, and sieving 100% erythritol ultrafine powders having an average particle diameter of from 0.4 μm to 23 μm while spraying ethanol alcohol thereon.

In the present invention, pherical erythritol granules for direct compression are obtained by granulating, drying, and sieving 100% erythritol ultrafine powders having an average particle diameter of from 0.4 μm to 23 μm while spraying ethanol alcohol thereon.

### BACKGROUND OF THE INVENTION

1. Field of the Invention

The present invention relates to spherical erythritol granules obtained by directly compressing an aggregate of 100% erythritol ultrafine powders having an average particle diameter of from 0.1 to 23 μm in a hydrous alcohol atmosphere and a process of producing the spherical erythritol granules, and tablets having the erythritol ingredient and either a pharmaceutically effective ingredient or a nutritional ingredient.

2. Background Art

As conventional erythritol-containing disintegrating tablets, Patent Document 1 describes an invention of a disintegrating tablet containing a medicament having an equilibrium water content of from 10 to 20% at 25° C. and a relative humidity of 75%, erythritol, mannitol, and hydroxypropyl cellulose having an equilibrium water content of from 5 to 20% at 25° C. and a relative humidity of 75% and having, as a 20 mass % aqueous solution, a viscosity at 37° C. of from 100 to 2000 cps, characterized in that the disintegrating tablet contains the medicament in an amount of from 15 to 50 mass % based on the total mass of the disintegrating tablet and has a porosity of from 10 to 40%.

As a conventional process of producing erythritol-containing spherical granules, Patent Document 2 describes an invention of a process of producing erythritol-containing spherical granules including adding ethanol to an erythritol-containing composition, kneading the mixture, granulating the kneaded mass by extrusion, spheronizing the granulate by using a high-speed mixer/stirrer/granulator, and then drying.
[Patent Document 1] Japanese Patent Laid-Open No. 2010-163428
[Patent Document 2] Japanese Patent Laid-Open No. 2000-119173

### SUMMARY OF THE INVENTION

In the invention of Patent Document 1, there is proposed a process of tableting wetted tablet raw materials. The process of Patent Document 1 however has such a drawback that it cannot be applied when a main ingredient is required to have stability. In addition, the process of Patent Document 1 needs various control steps such as control of a relative humidity, control of a medicament so that it has an equilibrium water content within a predetermined range, and control of a porosity of a disintegrating tablet. Thus, the process of the invention needs complex production steps and in addition, has a drawback in a production cost.

According to the description on the invention of Patent Document 2, in Example 1 (preparation of spherical granules), a paste is prepared by adding 70 g of ethanol to 285 g of erythritol (filtered through a 100-mesh sieve) and 15 g of citric anhydride (filtered through a 100-mesh sieve) and kneading the resulting mixture by using a Shinagawa universal mixer (refer to Column 0011 of the specification).

It is apparent from the description that erythritol is not used alone but is mixed with citric anhydride or ethanol. It is also apparent that erythritol and citric anhydride are both particles passing through a 100-mesh sieve and therefore are large particles with a particle diameter of 140 μm at the maximum. Erythritol having such a large particle diameter cannot constitute 100% erythritol granules.

This means that since erythritol has a considerably low solubility, it was impossible to prepare a granular product composed only of fine erythritol powders by using conventional erythritol having a particle diameter on the order of 100 μm. Even if granules are prepared using only of fine erythritol powders, the granular product thus obtained will collapse, making it difficult to industrialize such a granular product.

In consideration of the characteristic that ultrafine erythritol powders have a very low solubility, the present inventors have prepared ultrafine erythritol powders having an average particle diameter of from 0.4 to 23 μm to increase the surface area of the erythritol. This makes it possible to dissolve the surface of the ultrafine erythritol powders even in a trace amount of water. As a result, they have succeeded in production of granules composed only of erythritol.

The present invention has been made based on the above-mentioned conventional technology. It was conventionally difficult to produce 100% erythritol spherical granules for direct compression. The present invention however provides 100% erythritol spherical granules for direct compression, a process of producing these spherical granules, and tablets containing the 100% erythritol ingredient a pharmaceutically effective component and a nutritional component.

An object of the present invention is to provide 100% erythritol spherical granules for direct compression and a process of producing tablets including directly compressing the 100% erythritol spherical granules with a pharmaceutically effective ingredient or nutritional ingredient. This process can be used even when a main ingredient is not stable in water. It is provided in order to reduce the number of production steps and a production cost. In particular, when erythritol granules are used as a pharmaceutical additive, remaining of a binder other than erythritol granules in a granular product is not permitted at present so that an object of the invention is to carry out tableting using, together with a pharmaceutical additive, only erythritol granules as a binder.

The invention provided in claim 1 is spherical erythritol granules for direct compression obtained by granulating, drying, and sieving 100% erythritol ultrafine powders having an average particle diameter of from 0.4 μm to 23 μm while spraying ethanol alcohol thereon.

The invention provided in claim 2 is a process of producing spherical erythritol granules for direct compression including a step of injecting a 100% erythritol raw material from the injection tube by using a solid pulverizing and drying apparatus equipped with supports provided at intervals in a circumferential direction, on the side of a center portion, between a lower disk and an upper disk that horizontally turn at a high speed around a center axis in a circular road casing installed horizontally in the apparatus, blades provided on the side of the peripheral portion of the lower disk and the upper disk and at a position deviated from the supports while having intervals in a circumferential direction, and an injection tube provided vertically toward a hole provided at the center portion of the upper disk; a step of pulverizing the raw material by making use of the impact caused by collision between the raw material and each of the supports and the blades of the disks turning at a high speed; a step of classifying the pulverized erythritol into powders having an average particle diameter ranging from 0.4 μm to 23 μm; a step of granulating thus classified ultrafine erythritol powders by using a granulator; a step of drying the thus granulated erythritol; and a step of sieving/classifying the thus dried erythritol.

The invention according to claim 3 is a process of producing tablets including tableting the 100% erythritol granules as described in claim 1 with a pharmaceutically effective ingredient or a nutritional ingredient by using a direct compression method.

The invention according to claim 4 is an orally disintegrating tablet or chewable tablet as specified in the Japanese Pharmacopoeia, or a health food tablet or ordinary tablet candy that melts in the mouth obtained by the producing process of claim 3.

Due to the considerably low solubility of erythritol, it was impossible to prevent collapse and keep the shape of a granular product having fine powders of a conventional powder size as a constituting unit. In the present invention, on the other hand, as spherical erythritol powders having a considerably low solubility, only ultrafine erythritol powders having a particle size distribution within a range of an average particle diameter of from 0.4 μm to 23 μm are used. It improves the surface area of erythritol and makes it possible to dissolve the surface of the ultrafine erythritol powders in spite of a slight water content. Spherical granules can therefore be obtained using only ultrafine erythritol powders.

As a result, the spherical erythritol granules for direct compression according to the present invention have the following advantageous effects. In orally disintegrating tablets and chewable tablets in pharmaceuticals and health foods, they are expected to have a high dissolution rate in the mouth, provide a good taste, and have an effect of masking a bitter taste or strange taste of the main ingredient or health materials. In particular, they can improve a yield of erythritol powders having an average particle diameter not greater than 5 μm and as a result, spherical erythritol granules for direct compression can be obtained with a high productivity.

Among materials for granules used for orally disintegrating tablets or chewable tablets in pharmaceuticals and health foods, erythritol is judged as one of the most effective materials.

Tablets using erythritol have so far been produced and put on the market. There is proposed a process of tableting dampened tablet raw materials in “Orally disintegrating tablets” (Japanese Patent Laid-Open No. 2010-163428) and the like. Most of the conventional processes however cannot be used, depending on the stability of a main ingredient, or require complex production steps.

The 100% erythritol granular product for direct compression as proposed in the invention is mixed with a main ingredient and other additives without adding water and then compressing the resulting mixture into tablets so that it can be used without a problem even when the main ingredient is not stable in water. In addition, when this product is used, the number of production steps and a production cost can be reduced.

The present invention exhibits its effect fully when erythritol granules are used as a pharmaceutical additive and remaining of a binder other than erythritol in a granular product is not permitted. The invention has realized the development of 100% erythritol spherical granules for direct compression which has been long waited.

According to the present invention, 100% erythritol spherical granules for direct compression can be put on the market. The present invention has more characteristic effects. Described specifically, disintegrating tablets or chewable tablets obtained using these 100% erythritol spherical granules have a hardness of 40N or greater and have a disintegration rate within 30 seconds, do not cause tableting problems such as capping, and have a friability not greater than 0.5%.

In the process of producing spherical erythritol granules for direct compression according to the present invention, an air flow pulverizer in which an erythritol raw material is pulverized as a result of collision with each of supports and blades caused by the centrifugal force of a rapidly rotating disk is employed. Different from a conventional air flow pulverizer using a jet mill, the above-mentioned air flow pulverizer can pulverize the erythritol raw material into ultrafine particles having an average particle diameter not greater than 20 μm. Since such ultrafine particles can be obtained in a yield as high as 99% or greater, the process of the present invention can have a greatly improved productivity and at the same time, can provide ultrafine erythritol powders with good properties.

### BRIEF DESCRIPTION OF THE DRAWINGS

FIG. 1 is a control flow chart of erythritol granules according to the present invention.

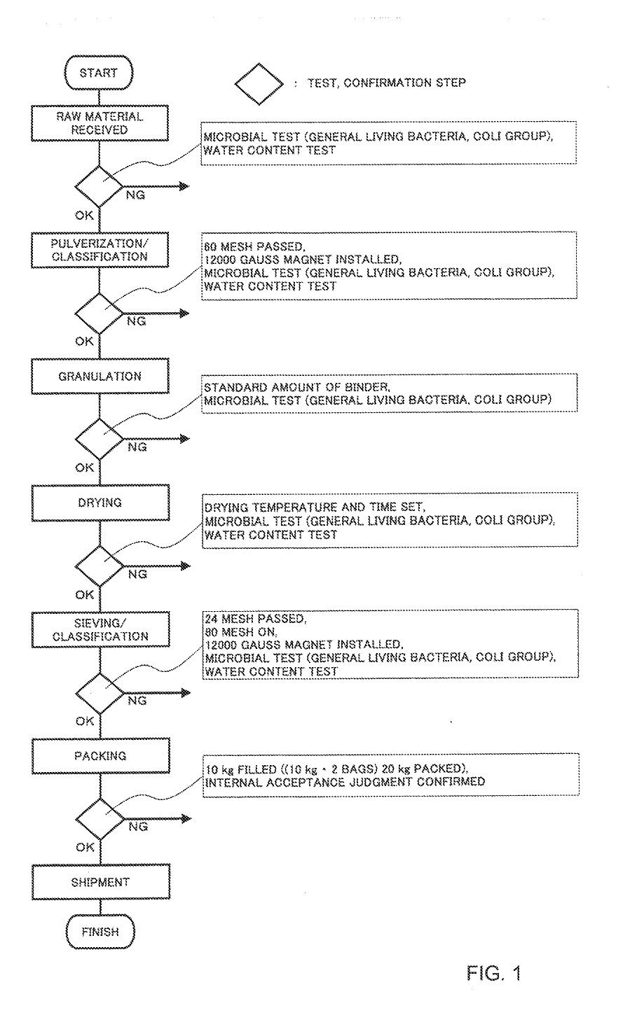

FIG. 2 shows a particle diameter distribution of erythritol according to the present invention.

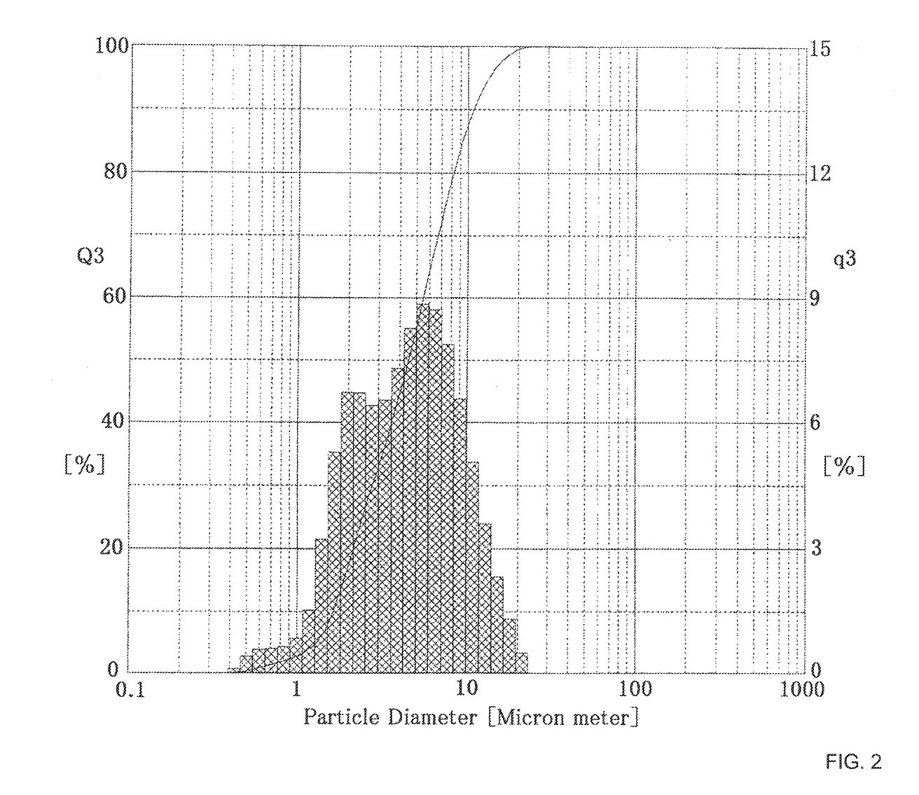

FIG. 3 is an overall side cross-sectional view of a solid pulverizing and drying apparatus to be used for pulverization of an erythritol raw material according to the present invention.

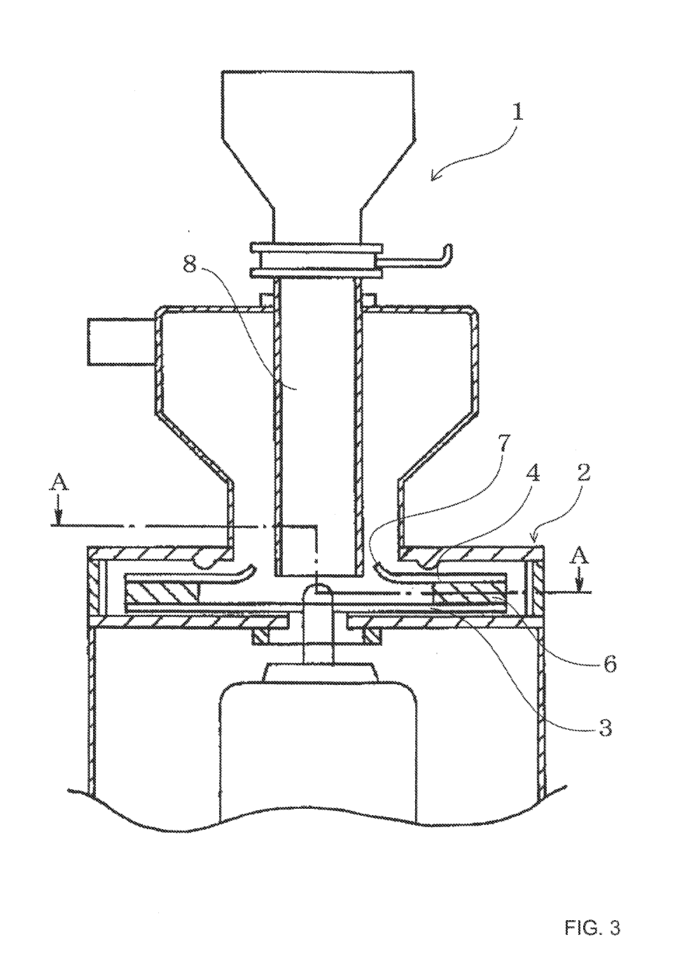

FIG. 4 is a cross-sectional view taken along an A-A arrow shown in FIG. 3.

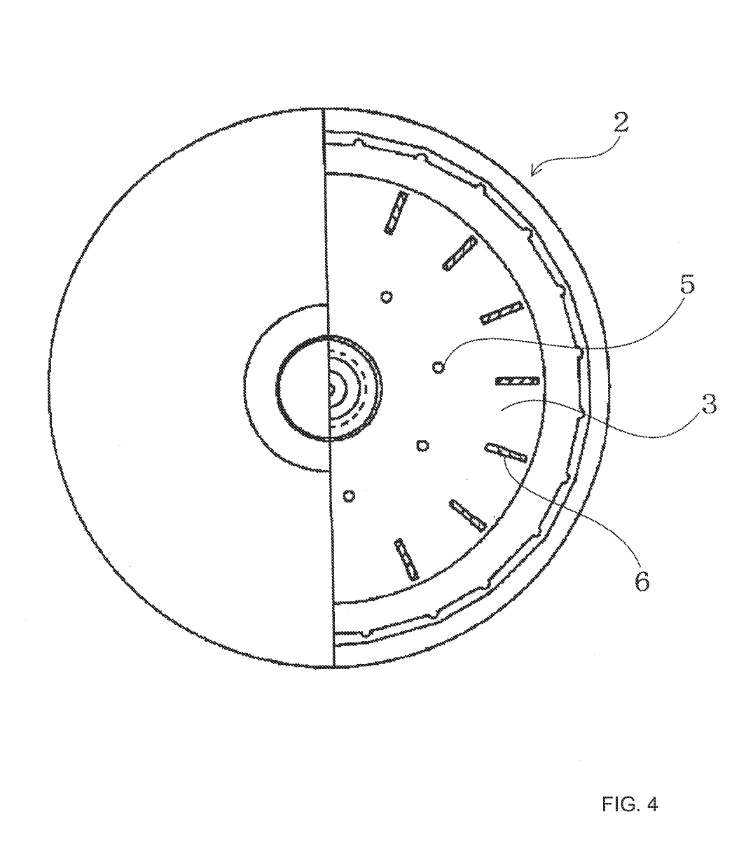

FIG. 5 is a partial side cross-sectional view of a rotary impeller portion of FIG. 3.

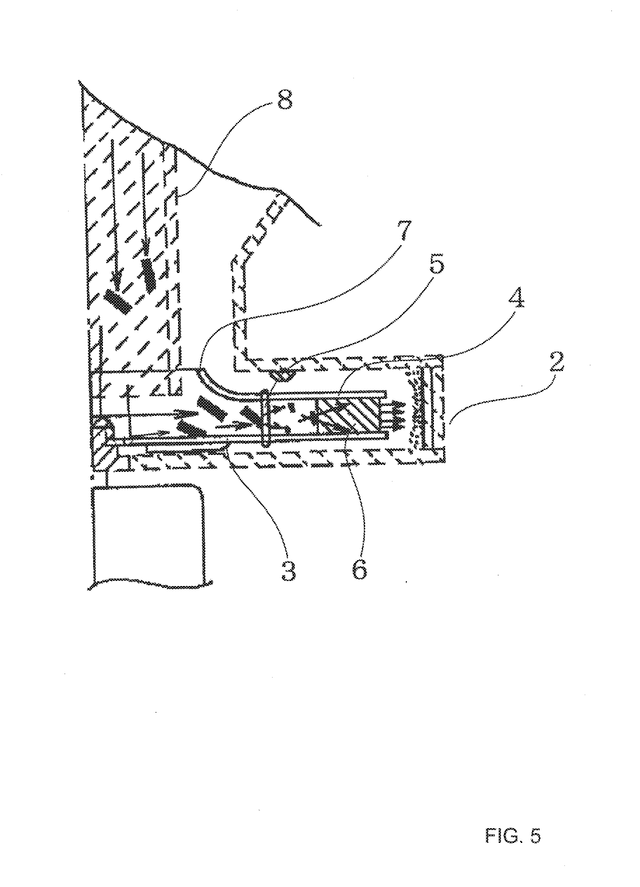

FIG. 6 is a cross-sectional view of a plane portion of the rotary impeller shown in FIG. 3.

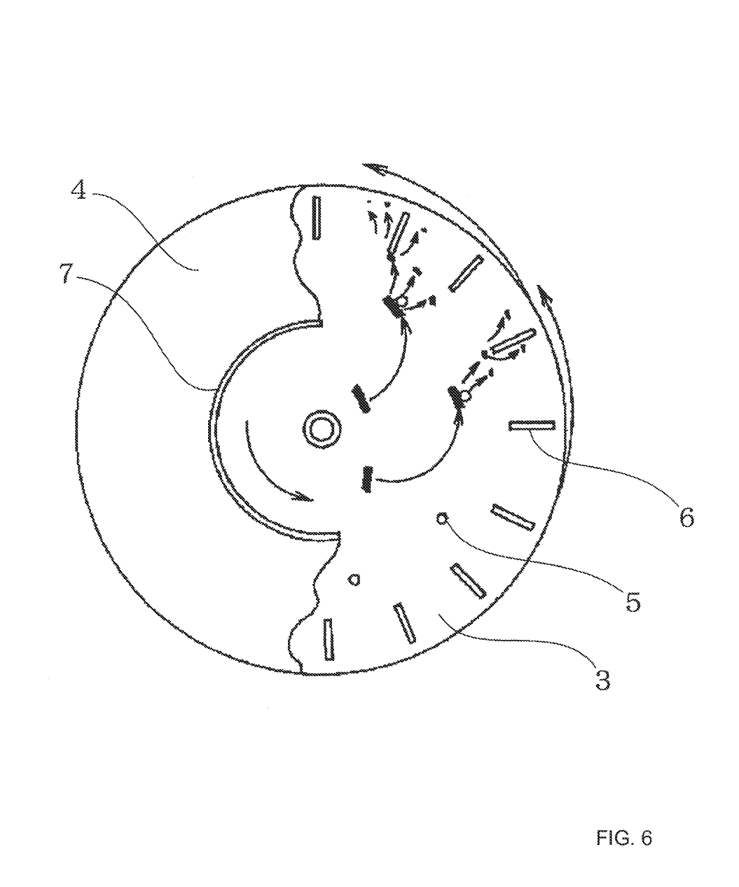

FIG. 7 is a partially enlarged side cross-sectional view showing the rotary impeller portion of FIG. 3.

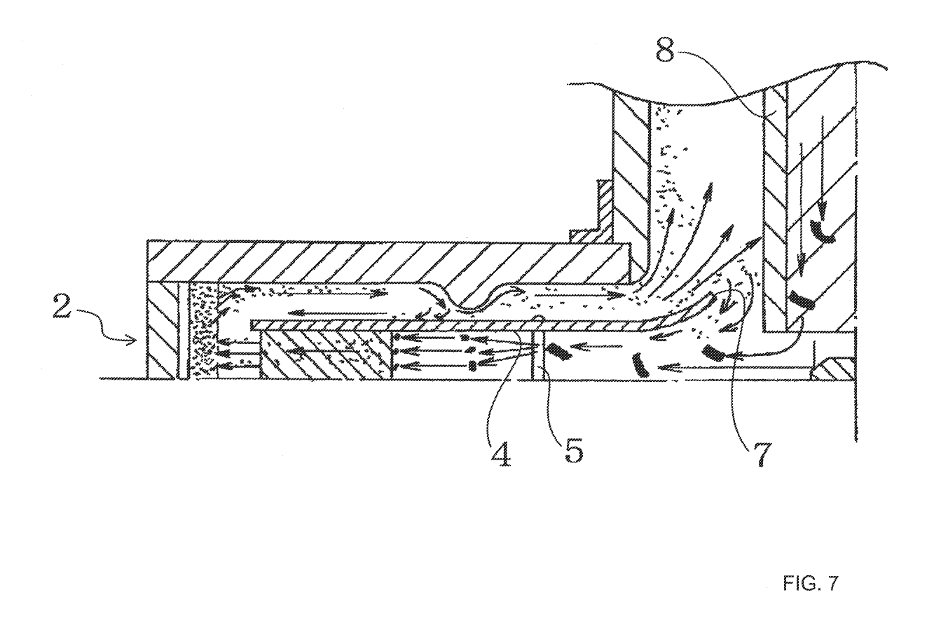

FIG. 8 is a plan view schematically showing a polyhedral shape of a circular road casing and the rotary impeller for describing an air flow generated by the circular impeller of FIG. 3.

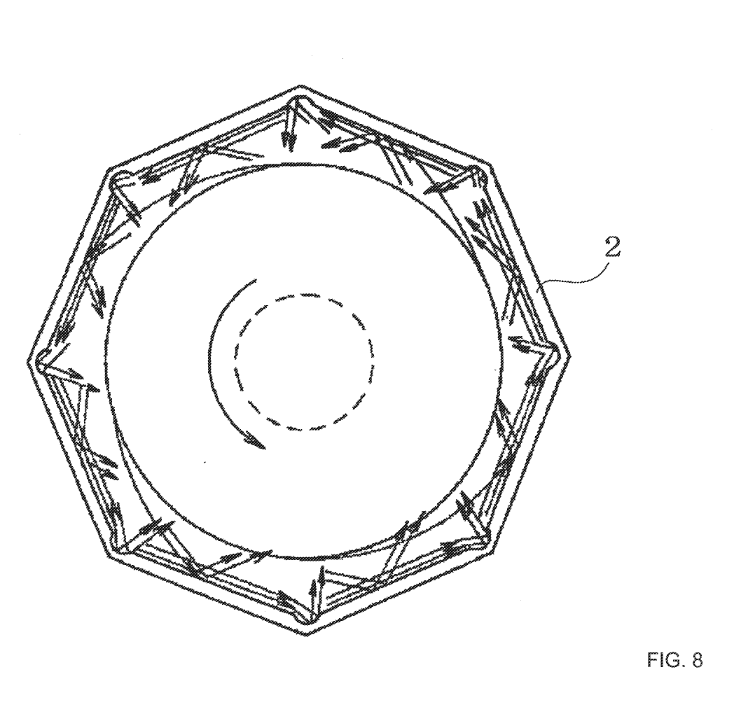

### DESCRIPTION OF THE PREFERRED EMBODIMENTS

### Example 1

A description will next be made based on the control flow chart 1 of the erythritol granules according to the present invention.

After receipt, a 100% erythritol raw material is subjected to a microbial test with respect to general living bacteria and a coli group and a water content test. These tests are followed by a pulverization/classification step. This pulverization/classification step is conducted using an air flow pulverizer 1. In this air flow pulverizer 1, supports 5 are provided with intervals in a circumferential direction on the side of a center portion between a lower disk 3 and an upper disk 4 that horizontally rotate at a high speed around a central axis in a horizontally installed circular load casing 2. The supports are installed at a position deviated from blades 6 provided on the side of a peripheral portion of the lower disk 3 and the upper disk 4 when viewed in a radius direction, but the supports 5 and the blades 6 are both provided at intervals in a circumferential direction. The 100% erythritol raw material is injected from an injection tube 8 installed vertically toward a hole 7 provided at the center portion of the upper disk 4. The 100% erythritol raw material injected from the injection tube 8 is caused to collide with each of the supports and the blades caused by a centrifugal force of the rapidly rotating disks and thereby pulverized. The erythritol thus pulverized is classified as an ultrafine powder having an average particle diameter of from 0.4 μm to 23 μm. From the ultrafine erythritol powder thus obtained, metal impurities are then removed by means of a 12000 gauss magnet. The resulting ultrafine erythritol powder is then subjected to a microbial test with respect to general living bacteria and a coli group, and a water content test.

In the present invention, the average particle diameter of erythritol is defined to fall within a range of from 0.4 μm to 23 μm, because ultrafine erythritol powders have an increased surface area and even at a considerably small water content, they bind to each other with an enhanced binding force irrespective of a slight water content. Powders having an average particle diameter less than 4 μm have difficulty in production control, while when powders have an average particle diameter exceeding 23 μm, granules are likely to collapse because of a weakened binding force among ultrafine erythritol powders.

After granulation, the granulated erythritol is dried for a predetermined time at a temperature not damaging the properties of erythritol to remove the water content. After removal of the water content, erythritol granules are subjected to a sieving/classification step. The erythritol granules are sieved into a substantially uniform size in this sieving step. After this sieving/classification step, magnetic metals are removed from the resulting sieved and classified erythritol granules by means of a 12000 gauss magnet. Then, a microbial test and a water content test are conducted. The erythritol granules which have passed these tests are packed and then shipped.

TABLE 1
Measurement results of particle size distribution

| | |
|---|---|
| Registration No | 41 |
| Sample name | For erythritol granules |
| Lot | |
| Dispersion medium | 0.3 Mpa |
| Dispersing agent | |
| Measurement time and date | 2013/01/16 10:01:15 |
| Measurer | Shoji |
| Remarks | |
| Refractive index | 1.330-0.001 |
| Shape factor | 1.000 |
| X10 | 1.644 μm |
| X50 | 4.532 μm |
| X90 | 10.862 μm |
| Specific surface area | 1.864 m²/cm³ |
| R•R—N | 1.8485 |
| R•R—B | 0.03273 |
| Normal distribution 50% | 4.066 μm |
| Normal distribution σg | 2.127 |
| Sample concentration | 198 mV |

TABLE 2
Measurement results of erythritol particle size distribution

| | Particle | | |
|---|---|---|---|
| | diameter | Cumulative | Interval |
| Ch. | μm | % | % |
| 1 | 0.10 | 0.00 | 0.00 |
| 2 | 0.12 | 0.00 | 0.00 |
| 3 | 0.14 | 0.00 | 0.00 |
| 4 | 0.17 | 0.00 | 0.00 |
| 5 | 0.20 | 0.00 | 0.00 |
| 6 | 0.23 | 0.00 | 0.00 |
| 7 | 0.28 | 0.00 | 0.00 |
| 8 | 0.33 | 0.00 | 0.00 |
| 9 | 0.39 | 0.00 | 0.00 |
| 10 | 0.46 | 0.10 | 0.10 |
| 11 | 0.55 | 0.51 | 0.41 |
| 12 | 0.65 | 1.08 | 0.57 |
| 13 | 0.77 | 1.68 | 0.60 |
| 14 | 0.92 | 2.32 | 0.64 |
| 15 | 1.09 | 3.17 | 0.85 |
| 16 | 1.29 | 4.70 | 1.53 |
| 17 | 1.53 | 7.93 | 3.23 |
| 18 | 1.81 | 13.23 | 5.30 |
| 19 | 2.15 | 19.95 | 6.72 |
| 20 | 2.55 | 26.66 | 6.71 |
| 21 | 3.02 | 33.07 | 6.41 |
| 22 | 3.58 | 39.61 | 6.54 |
| 23 | 4.24 | 46.91 | 7.30 |
| 24 | 5.03 | 55.18 | 8.27 |
| 25 | 5.97 | 64.03 | 8.85 |
| 26 | 7.07 | 72.74 | 8.71 |
| 27 | 8.39 | 80.61 | 7.87 |
| 28 | 9.95 | 87.19 | 6.58 |
| 29 | 11.79 | 92.25 | 5.06 |
| 30 | 13.98 | 95.85 | 3.60 |
| 31 | 16.58 | 96.18 | 2.33 |
| 32 | 19.66 | 99.50 | 1.32 |
| 33 | 23.31 | 100.00 | 0.50 |
| 34 | 27.64 | 100.00 | 0.00 |
| 35 | 32.78 | 100.00 | 0.00 |
| 36 | 38.86 | 100.00 | 0.00 |
| 37 | 46.08 | 100.00 | 0.00 |
| 38 | 54.64 | 100.00 | 0.00 |
| 39 | 64.79 | 100.00 | 0.00 |
| 40 | 76.82 | 100.00 | 0.00 |
| 41 | 91.09 | 100.00 | 0.00 |
| 42 | 108.01 | 100.00 | 0.00 |
| 43 | 128.07 | 100.00 | 0.00 |
| 44 | 151.86 | 100.00 | 0.00 |
| 45 | 180.07 | 100.00 | 0.00 |
| 46 | 213.51 | 100.00 | 0.00 |
| 47 | 253.17 | 100.00 | 0.00 |
| 48 | 300.19 | 100.00 | 0.00 |
| 49 | 355.95 | 100.00 | 0.00 |
| 50 | 422.06 | 100.00 | 0.00 |
| 51 | 500.45 | 100.00 | 0.00 |
| 52 | 593.40 | 100.00 | 0.00 |
| 53 | 703.61 | 100.00 | 0.00 |
| 54 | 834.27 | 100.00 | 0.00 |
| 55 | 1000.00 | 100.00 | 0.00 |

In the present invention, it is only necessary to mix a 100% erythritol granular product for direct compression with a main ingredient and other additives without adding water and then compress the resulting mixture into tablets. Even when the main ingredient is not stable in water, the granular product can be used without a problem so that the present invention can be used widely in various industrial fields including health food industry and pharmaceutical industry.
1: Air-flow pulverizer
2: Circular load casing
3: Lower disk
4: Upper disk
5: Support
6: Blade
7: Hole
8: Injection tube

1. Spherical erythritol granules for direct compression obtained by granulating, drying, and sieving 100% erythritol ultrafine powders having an average particle diameter of from 0.4 μm to 23 μm while spraying ethanol alcohol thereon.

2. A process of producing spherical erythritol granules for direct compression including a step of injecting a 100% erythritol raw material from the injection tube by using a solid pulverizing and drying apparatus equipped with supports provided at intervals in a circumferential direction, on the side of a center portion, between a lower disk and an upper disk that horizontally turn at a high speed around a center axis in a circular road casing installed horizontally in the apparatus, blades provided on the side of the peripheral portion of the lower disk and the upper disk and at a position deviated from the supports while having intervals in a circumferential direction, and an injection tube provided vertically toward a hole provided at the center portion of the upper disk and; a step of pulverizing the raw material by making use of the impact caused by collision between the raw material and each of the supports and the blades of the disks turning at a high speed; a step of classifying the pulverized erythritol into powders having an average particle diameter ranging from 0.4 μm to 23 μm; a step of granulating thus classified ultrafine erythritol powders by using a granulator; a step of drying the thus granulated erythritol; and a step of sieving/classifying the thus dried erythritol.

3. A process of producing tablets including tableting the 100% erythritol granules as described in claim 1 with a pharmaceutically effective ingredient or a nutritional ingredient by using a direct compression method.

4. An orally disintegrating tablet or chewable tablet as specified in the Japanese Pharmacopoeia, or a health food tablet or ordinary tablet candy that melts in the mouth obtained by the producing process of claim 3.
